(19) United States
(12) Patent Application Publication
(10) Pub. No.: US 2010/0168329 A1
(43) Pub. Date: Jul. 1, 2010

(54) ALIPHATIC, CYCLOALIPHATIC OR (CYCLO)ALIPHATIC DIISOCYANATES THAT ARE STABLE IN STORAGE

(75) Inventors: Dirk Hoppe, Nottuln (DE); Rainer Lomoelder, Muenster (DE); Stephan Kohlstruk, Duelmen (DE); Hans-Werner Michalczak, Herne (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: DEGUSSA GmbH, Duesseldorf (DE)

(21) Appl. No.: 11/720,812

(22) PCT Filed: Oct. 14, 2005
(86) PCT No.: PCT/EP05/55271

§ 371 Date: Jun. 4, 2007

(30) Foreign Application Priority Data
Dec. 2, 2004 (DE) 10 2004 058 173.8

(51) Int. Cl. C07C 249/00; C08L 75/00

(52) U.S. Cl. 524/871; 560/331; 560/332; 560/333

(57) ABSTRACT

The invention relates to aliphatic, cycloaliphatic or (cyclo)aliphatic diisocyanates, in addition to a method for their production.

The invention relates to storage-stable aliphatic, cycloaliphatic or (cyclo)aliphatic diisocyanates and to a process for their preparation.

Organic polyisocyanates, for example aromatic, cycloaliphatic, (cyclo)aliphatic and aliphatic difunctional and higher-functionality polyisocyanates are prepared industrially typically by reacting the corresponding amines with phosgene (phosgenation) and the cleavage of the resulting polycarbamoyl chlorides.

Problems in this procedure are the high conversion of chlorine via phosgene and carbamoyl chloride to hydrogen chloride, the toxicity of phosgene and the associated costly safety precautions, the corrosivity of the reaction mixture, the lability of the solvents typically used and the formation of chlorinated and chlorine-free by-products, that have an impact on physical properties, for example the color, viscosity and vapor pressure, and on chemical properties, for example reactivity, storage stability, inter alia, of the polyisocyanates, and on the mechanical properties of the polyisocyanate polyaddition products prepared from such polyisocyanates.

Alternatively, diisocyanates such as hexamethylene 1,6-diisocyanate (HDI), 2,2,4-trimethyl-hexamethylene 1,6-diisocyanate and its 2,4,4-trimethyl isomer (TMDI), and 1-isocyanato-3-isocyanatomethyl-3,5,5-trimethylcyclohexane (isophorone diisocyanate, IPDI) can be prepared according to EP 126 299 (U.S. Pat. No. 4,596,678), EP 126 300 (U.S. Pat. No. 4,596,679), EP 355 443 (U.S. Pat. No. 5,087,739) and EP 568 782 (U.S. Pat. No. 5,360,931) in a circulation process by reacting the corresponding diamines with urea and alcohols, and optionally N-unsubstituted carbamic esters, dialkyl carbonates and other by-products recycled from the reaction process, to give biscarbamic esters, and their thermal cleavage to the corresponding diisocyanates and alcohols.

In addition, diisocyanates, for example IPDI, ring-hydrogenated MDI (H₁₂MDI) and ring-hydrogenated TDI (H₆TDI) can be prepared using dimethyl carbonate via similar technology to diisocyanates described above, likewise avoiding chlorine as a raw material (EP 976 723).

The storage stability, the reactivity and the color of the diisocyanates and the products prepared therefrom depend on by-products, some of unknown structure, which are present in the diisocyanates. The type and amount of these by-products depend upon the preparation process. It has been found that especially the chlorinated by-products occurring in the phosgenation influence the storage stability, the reactivity and color of the products prepared from the diisocyanates.

According to information given in U.S. Pat. No. 3,330,849, organic polyisocyanates, for example, can be stabilized against discoloration and precipitate formation by the addition of sulfonyl isocyanates. The addition of metal naphthenates, for example cadmium naphthenate, cobalt naphthenate, copper naphthenate, lead naphthenate, manganese naphthenate or zinc naphthenate allows the hydrolyzable chlorine content of isocyanates to be reduced according to U.S. Pat. No. 3,373,182. U.S. Pat. No. 3,384,653 and U.S. Pat. No. 3,449,256 describe the improvement in the storage stability of diphenylmethane 4,4-diisocyanate by a treatment at from 160 to 250° C. with trialkyl phosphates. According to U.S. Pat. No. 3,458,558, the content of hydrolyzable chlorine compounds can be lowered in the case of organic isocyanates also with copper, silver, nickel, iron and zinc at temperatures above 100° C. According to the information of U.S. Pat. No. 3,479,393, trialkylaminoboranes stabilize isocyanates against discoloration. According to U.S. Pat. No. 3,535,359, orthocarboxylic esters are suitable for stabilizing organic isocyanates against viscosity increase. According to the information of U.S. Pat. No. 3,585,229, polyisocyanate mixtures comprising diphenylmethane diisocyanate can be decolorized by addition of diphenyldecyl phosphate. It is possible to stabilize organic polyisocyanates according to U.S. Pat. No. 3,692,813 against decomposition with the aid of oxycarbonyl isocyanates having at least one group of the formula —O—CO—NCO. For the stabilization of organic polyisocyanates against discoloration, it is possible according to the information of U.S. Pat. No. 3,715,381 to use 2,6-di-tert-butyl-p-cresol. According to U.S. Pat. No. 3,970,680, diphenylmethane diisocyanates can also be stabilized by addition of tertiary amines. To purify organic isocyanates, they can be treated according to U.S. Pat. No. 4,065,362 at temperatures above 100° C. with a metal salt of mercaptobenzothiazole, with a metal salt of alkyl-substituted dithiocarbamic acids, with an alkyl-substituted phenol, with a thiobisphenol or with a triaryl phosphite. According to the information of U.S. Pat. No. 3,247,236, it is possible to stabilize diisocyanates, prepared by reaction of diamines with phosgene and purified by distillation, by addition of carbon dioxide or sulfur dioxide. A disadvantage of this process is the good solubility of sulfur dioxide in the polyisocyanate and the formation of discolorations during storage. The patent publications mentioned impart no teaching with regard to the stabilization of organic polyisocyanates prepared by phosgene-free processes, preferably of organic polyisocyanates prepared by thermal cleavage of organic polycarbamic esters.

One disadvantage of the aliphatic, cycloaliphatic or (cyclo)aliphatic diisocyanates prepared by the above-described phosgene-free processes is their tendency, in the course of storage, to form linear polymers having the following (nylon-1) structure

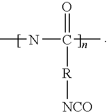

In the case of hexamethylene diisocyanate (HDI), these polymers can lead to gelling, while products prepared from trimethylhexamethylene diisocyanate (TMDI) can exhibit undesired opacity.

DE 43 31 085 describes the stabilization of aliphatic, cycloaliphatic or (cyclo)aliphatic diisocyanates prepared by phosgene-free processes by use of carbon dioxide as a stabilizer.

EP 643 042 describes the stabilization of aliphatic, cycloaliphatic or (cyclo)aliphatic diisocyanates prepared by phosgene-free processes. The stabilizers described are compounds which serve as antioxidants and/or radical scavengers (primary stabilizers), which act as peroxide cleavers and/or reducing agents (secondary stabilizers), and acidic compounds (acidic stabilizers) or mixtures of the individual stabilizer groups.

Primary stabilizers in the sense of EP 643 042 alone are not suitable as stabilizers because they promote the oligomerization of aliphatic, cycloaliphatic or (cyclo)aliphatic diisocyanates prepared by a phosgene-free process, as we have determined.

It is therefore an object of the invention to stabilize aliphatic, cycloaliphatic and (cyclo)aliphatic diisocyanates prepared by a phosgene-free process with regard to oligomerization and color.

Surprisingly, this object is achieved by treating the aliphatic, cycloaliphatic and (cyclo)aliphatic diisocyanates with dry air and/or dry synthetic air and/or dry oxygen which are bubbled through the aliphatic, cycloaliphatic or (cyclo)aliphatic diisocyanates during or preferably directly after the synthesis. If appropriate, further stabilizers known per se may be used in addition.

The invention provides storage-stable aliphatic, cycloaliphatic and (cyclo)aliphatic diisocyanates and mixtures thereof, prepared by a phosgene-free process, which comprise oxygen for stabilization.

The oxygen is introduced into the diisocyanate or into the diisocyanate mixture preferably after the diisocyanate synthesis. It is also possible in principle to carry out the phosgene-free synthesis of the aliphatic, cycloaliphatic or (cyclo)aliphatic diisocyanates actually in the presence of oxygen.

The storage-stable aliphatic, cycloaliphatic or (cyclo)aliphatic diisocyanates comprise, at 25° C. and standard pressure, from 1 to 300 ppm (from 0.0007 to 0.21 mol %) of oxygen, preferably from 3 to 200 ppm (from 0.002 to 0.14 mol %) of oxygen, more preferably from 5 to 100 ppm (from 0.004 to 0.07 mol %) of oxygen, and the oxygen may be introduced by means of pure dry oxygen and/or dry air and/or dry synthetic air. It is also possible that further inert gases such as nitrogen and/or noble gases are present in oxygen.

It has also been found to be useful to blanket the diisocyanate or the diisocyanate mixture additionally with an oxygenous atmosphere in a storage vessel example storage tank or vat).

The diisocyanate compositions according to the invention may comprise any aliphatic,

cycloaliphatic and (cyclo)aliphatic diisocyanates, with the proviso that they are prepared by suitable processes in the absence of phosgene. Preferred diisocyanates have been found to be, and preference is therefore given to using, aliphatic, cycloaliphatic and (cyclo)aliphatic diisocyanates which are obtainable by thermal cleavage of aliphatic, cycloaliphatic and (cyclo)aliphatic dicarbamic esters. Suitable aliphatic diisocyanates have advantageously from 3 to 16 carbon atoms, preferably from 4 to 12 carbon atoms, in the linear or branched alkylene moiety, and suitable cycloaliphatic or (cyclo)aliphatic diisocyanates have advantageously from 4 to 18 carbon atoms, preferably from 6 to 15 carbon atoms, in the cycloalkylene radical. Examples include: 1,4-diisocyanotobutane, 2-ethyl-1,4-diisocyanatobutane, 1,5-diisocyantopentane, 2,2-dimethyl-1,5-diisocyanatopentane, 2-methyl-1,5-diisocyanatopentane (MPDI), 2-ethyl-2-propyl-1,5-diisocyanatopentane, 2-ethyl-2-butyl-1,5-diisocyanatopentane, 2-alkoxy-1,5-diisocyanatopentane, hexamethylene 1,6-diisocyanate (HDI), 2,4,4- or 2,2,4-trimethylhexamethylene 1,6-diisocyanate (TMDI), 1,7-diisocyanatoheptane, 1,8-diisocyanatooctane, 1,10-diisocyanatodecane, 1,12-diisocyanatododecane, 4,4′-diisocyanatodicyclohexylmethane, 2,4′-diisocyanatodicyclohexyimethane and mixtures of the isomeric diisocyanatodicyclohexylmethanes (H12MDI), 1,3-diisocyanatocyclohexane, and isomer mixtures of diisocyanatocyclohexanes and 1-isocyanato-3-isocyanatomethyl-3,5,5-trimethylcyclohexane (IPDI).

The aliphatic, cycloaliphatic and (cyclo)aliphatic diisocyanates used are preferably 1-isocyanato-3-isocyanatomethyl-3,5,5-trimethylcyclohexane, 2,4,4- or 2,2,4-trimethylhexamethylene 1,6-diisocyanate, 4,4′-diisocyanatodicyclohexylmethane, 2,4′-diisocyanato-dicyclohexylmethane and mixtures of the isomeric diisocyanatodicyclohexylmethanes and hexamethylene 1,6-diisocyanate.

Particular preference is given to IPDI, H₁₂MDI, TMDI, HDI and/or MPDI being present.

As already detailed, the aliphatic, cycloaliphatic and (cyclo)aliphatic diisocyanates are preferably prepared by thermal cleavage of the corresponding dicarbamic esters. This cleavage may be carried out, for example, at temperatures of from 150 to 300° C., preferably from 180 to 250° C., and pressures of from 0.001 to 2 bar, preferably from 1 to 200 mbar, in the absence or preferably the presence of catalysts in suitable cleavage reactors, for example thin-film evaporators, falling-film evaporators or heating cartridge evaporators according to EP 524 554. The diisocyanates and alcohols formed in the cleavage can be separated, for example, by fractional condensation or preferably by rectification, and diisocyanates can be additionally purified, for example, by distillation.

The inventively stabilized aliphatic, cycloaliphatic and (cyclo)aliphatic diisocyanates prepared by a phosgene-free process may be stabilized by dry air or oxygen alone. In addition, other stabilizing compounds may be used.

Suitable additional stabilizers against discoloration are, for example, primary stabilizers which are typically active as antioxidants and/or as radical scavengers. Primary stabilizers in the context of this invention are, for example, phenolic antioxidants which contain at least one sterically hindered phenolic moiety. Examples of these phenolic antioxidants are: 2,6-di-tert-butyl-4-methylphenol, 2,4,6-tri-tert-butylphenol, 2,2′-methylenebis(4-methyl-6-tert-butylphenol), 2,2′-thiobis(4-methyl-6-t-butylphenol), 4,4′-thiobis(3-methyl-6-t-butylphenol), 4,4′-butylidenebis(3-methyl-6-tert-butylphenol), 4,4′-methylidenebis(2,6-di-tert-butylphenol), 2,2′-methylidenebis[4-methyl-6-(1-methylcyclohexyl)phenol], tetrakis [methylene 3-(3,5-di-tert-butyl-4-hydroxyphenyl)propionate]methane, 1,3,5-trimethyl-2,4,6-tris(3,5-di-tert-butyl-4-hydroxybenzyl)benzene, N,N′-hexamethylenebis(3,5-di-tert-butyl-4-hydroxyhydrocinnamide), octadecyl 3-(3,5-di-tert-butyl-4-hydroxyphenyl)propionate, 1,3,5-tris(3,5-di-tert-butyl-4-hydroxybenzyl)isocyanurate, 1,1,3-tris(5-tert-butyl-4-hydroxy-2-methylphenyl)-butane, 1,3,5-tris-(3,5-di-tert-butyl-4-hydroxybenzyl)mesitylene; ethylene glycol bis[3,3-bis(3′-tert-butyl-4′-hydroxyphenyl)butyrate], 2,2′-thiodiethyl bis-3-(3,5-di-tert-butyl-4-hydroxyphenyepropionate, 2,2′-methylenebis(4-methyl-6-cyclohexylphenol), 1,6-hexanediol bis(3,5-di-tert-butyl-4-hydroxyphenyppropionate, 2,4-bis(n-octylthio)-6-(4-hydroxy-3,5-di-tert-butylanilino)-1,3,5-triazine, diethyl 3,5-di-tert-butyl-4-hydroxybenzylphosphonate and triethylene glycol bis-3-(tert-butyl-4-hydroxy-5-methylphenyl)propionate.

Preference is given to using octadecyl 3-(3,5-di-tert-butyl-4-hydroxyphenyl)propionate, 2,2′-methylenebis(4-methyl-6-cyclohexylphenol), 2,2′-methylenebis(4-methyl-6-tert-butylphenol), triethylene glycol bis-3-(tert-butyl-4-hydroxy-5-methylphenyl)propionate, tetrakis[methylene 3-(3,5-di-tert-butyl-4-hydroxyphenyl)propionate]methane and 2,6-di-tert-butyl-4-methyl-phenol.

The primary stabilizers are used in an amount of from 1 to 300 ppm, preferably in an amount of from 1 to 200 ppm, based on the weight of the diisocyanate composition.

Additionally used as stabilizers may be secondary stabilizers which are typically active as peroxide cleavers and/or reducing agents. Suitable secondary stabilizers are, for example, phosphorus compounds, preferably triesters of phosphorous acid, for example trialkyl phosphites and triaryl phosphites and thioethers.

Examples of the esters of phosphorous acid are distearylpentaerythritol diphosphite, tris(nonylphenyl)phosphite, tris(2,4-di-tert-butylphenyl)phosphite, neopentyl glycol triethylene glycol diphosphite, diisodecylpentaerythritoi diphosphite, tristearyl phosphite, trilauryl phosphite and in particular triphenyl phosphite.

Examples of the thioethers are 2-methyl-1-propenyl tert-dodecyl thioether, cyciohexylidene-methyl n-dodecyl thioether, 2-cyclohexen-1-ylidenemethyl n-octadecyl thioether, 2-cyclohexen-1-ylidenemethyl n-dodecyl thioether, 2-cyclohexen-1-ylidenemethyl n-octyl-thioether, 2-cyclohexen-1-ylidenemethyl n-cyclohexyl thioether, 2-cyclohexen-1-ylidenemethyl p-tolyl thioether and 2-cyclohexen-1-ylidenemethyl benzyl thioether.

The secondary stabilizers are used in an amount of from 1 to 300 ppm, preferably in an amount of from 1 to 200 ppm, based on the weight of the diisocyanate composition.

In a further preferred embodiment of the invention, from 1 to 200 ppm (from 0.0007 to 0.14 mol %) of oxygen and from 1 to 200 ppm (from 0.0002 to 0.02 mol %) of a primary stabilizer a) for color reduction are present under standard conditions. In a further preferred embodiment of the invention, from 5 to 100 ppm (from 0.0007 to 0.07 mol %) of oxygen and from 1 to 150 ppm (from 0.0002 to 0.015 mol %) of 2,6-di-tert-butyl-4-methylphenol are present under standard conditions.

In addition to the oxygen, it is also possible to use compositions which are obtained by partial reaction of oxygen with primary and/or secondary stabilizers. Further preferred embodiments of the invention are: TMDI stabilized with oxygen and/or air; TMDI stabilized with oxygen and/or with air and a trialkyl phosphite, in particular composed of tributyl phosphite and/or triphenyl phosphite; H₁₂MDI stabilized with oxygen and/or with air, H₁₂MDI stabilized with oxygen and/or with air and 2,6-di-tert-butyl-4-methylphenol, IPDI stabilized with oxygen and/or air, IPDI and 2,6-di-tert-butyl-4-methylphenol stabilized with oxygen and/or with air.

The invention also provides a process for preparing storage-stable aliphatic, cycloaliphatic or (cyclo)aliphatic diisocyanates, prepared by a phosgene-free process, which comprise oxygen for stabilization, the oxygen being introduced by means of passing dry air and/or dry oxygen into the aliphatic, cycloaliphatic or (cyclo)aliphatic diisocyanate.

The dry air and/or the dry oxygen is preferably introduced with a nozzle, a frit or by blanketing a turbulent flow into the diisocyanate, particular preference being given to introducing the air and/or the oxygen after the purifying distillation step of the diisocyanate synthesis. The air and/or the oxygen is introduced at temperatures of −20° C. to 200° C., at a pressure of from 5 mbar to 15 bar, with a volume flow rate of from 0.001 to 100 000 liters per hour.

Moreover, further inert gases, for example nitrogen or noble gases, for example argon, may additionally be present.

The invention also provides for the use of storage-stable aliphatic, cycloaliphatic or (cyclo)aliphatic diisocyanates and their subsequent products, for example allophanates, uretdiones, biurets, isocyanurates and prepolymers as coating composition raw materials and adhesive raw materials, and in particular for the use of storage-stable aliphatic, cycloaliphatic or (cyclo)aliphatic diisocyanates and their subsequent products, for example allophanates, uretdiones, biurets, isocyanurates and prepolymers in aqueous or solvent-containing, liquid or powder coating compositions.

The examples which follow are intended to further illustrate the invention.

### EXAMPLES

The extent of the opacities observed for some subsequent products of the 2,2,4-(2,4,4)-trimethylhexamethylene diisocyanate (TMDI) prepared by a phosgene-free process correlate outstandingly with the tendency to opacity of 10% solutions of the TMDI in acetonitrile or propylene carbonate, so that it was possible to employ these solutions as a rapid test for assessing the stability of the TMDI. The tendency to opacity of the subsequent products increases with increasing storage time of the TMDI. The opacity of the 10% solutions of TMDI in acetonitrile or propylene glycol can be determined quantitatively with the aid of a transmission measurement between 350 and 900 nm based on DIN EN 1557 (LICO 200 from Dr. Lange).

### Comparative Example 1

100 g of unstabilized 2,2,4-(2,4,4)-trimethylhexamethylene diisocyanate (TMDI) prepared by a phosgene-free process were dissolved directly after production in 900 g of propylene carbonate. The mixture was visually clear and had a transmission of 100% at a wavelength of 550 nm. Further samples of the TMDI were stored at room temperature and dissolved in a ratio of 1:9 in propylene glycol after four, six, eight and ten months. The results of the transmission measurements are compiled in Table 1. The transmission was measured in each case one hour after dissolution of the TMDI in propylene glycol.

### Example 1

Directly after preparation, dry synthetic air was passed at a temperature of 25° C. with a volume flow rate of 5 l per hour via a frit through 100 g of 2,2,4-(2,4,4)-trimethylhexamethylene diisocyanate (TMDI) prepared by a phosgene-free process. The saturation concentration of the oxygen in the TMDI was 0.025 mol % under these conditions. The thus stabilized TMDI was dissolved in propylene glycol and stored correspondingly to the comparative example and dissolved in propylene glycol after the times specified in the table which follows. The transmission was measured in each case one hour after dissolution of the TMDI in propylene glycol. The table which follows summarizes the results of the inventive example and of the comparative example:

| | |
|---|---|
| Storage time | Transmission [%] |

| [months] | Comparative Example 1 | Example 1 |
|---|---|---|
| 0 | 100 | 100 |
| 4 | 75 | 95 |
| 6 | 70 | 88 |
| 8 | 60 | 84 |
| 10 | 50 | 75 |

### Comparative Example 2

100 g of unstabilized 2,2,4-(2,4,4)-trimethylhexamethylene diisocyanate (TMDI) prepared by a phosgene-free process and stabilized with 100 ppm of 2,6-di-tert-butyl-4-methylphenol were dissolved directly after production in 900 g of propylene carbonate. The mixture was visually clear and had a transmission of 100% at a wavelength of 550 nm. Further samples of the TMDI stabilized with 100 ppm of 2,6-di-tert-butyl-4-methylphenol were stored at room temperature and dissolved in a ratio of 1:9 in propylene glycol after four, six, eight and ten months. The results of the transmission measurements are compiled in Table 1. The transmission was measured in each case one hour after dissolution of the TMDI in propylene glycol.

### Example 2

Directly after preparation, dry synthetic air was passed at a temperature of 25° C. with a volume flow rate of 5 l per hour via a frit through 100 g of 2,2,4-(2,4,4)-trimethylhexamethylene diisocyanate (TMDI) prepared by a phosgene-free process and stabilized with 100 ppm of 2,6-di-tert-butyl-4-methylphenol. The saturation concentration of the oxygen in the TMDI was 0.025 mol % under these conditions. The thus stabilized TMDI was dissolved in propylene glycol and stored correspondingly to the comparative example and dissolved in propylene glycol after the times specified in the table which follows. The transmission was measured in each case one hour after dissolution of the TMDI in propylene glycol. The table which follows summarizes the results of the inventive example and of the comparative example:

| | |
|---|---|
| Storage time | Transmission [%] |

| [months] | Comparative Example 2 | Example 2 |
|---|---|---|
| 0 | 100 | 100 |
| 4 | 40 | 80 |
| 6 | not measurable | 70 |
| 8 | not measurable | 60 |

1. Storage-stable aliphatic, cycloaliphatic or (cyclo)aliphatic diisocyanates and mixtures thereof, prepared by a phosgene-free process,
which comprise oxygen for stabilization.

2. The storage-stable aliphatic, cycloaliphatic or (cyclo)aliphatic diisocyanates as claimed in claim 1,
which comprise from 1 to 300 ppm (from 0.0007 to 0.21 mol %) of oxygen at 25° C. and standard pressure.

3. The storage-stable aliphatic, cycloaliphatic or (cyclo)aliphatic diisocyanates as claimed in claim 2,
which comprise from 3 to 200 ppm (from 0.002 to 0.14 mol %) of oxygen at 25° C. and standard pressure.

4. The storage-stable aliphatic, cycloaliphatic or (cyclo)aliphatic diisocyanates as claimed in claim 3,
which comprise from 5 to 100 ppm (from 0.004 to 0.07 mol %) of oxygen at 25° C. and standard pressure.

5. The storage-stable aliphatic, cycloaliphatic or (cyclo)aliphatic diisocyanates as claimed in claim 1,
wherein
the oxygen has been introduced by means of pure dry oxygen and/or dry air and/or dry synthetic air.

6. The storage-stable aliphatic, cycloaliphatic or (cyclo)aliphatic diisocyanates as claimed in claim 1,
which also contain inert nitrogen and/or noble gases.

7. The storage-stable aliphatic, cycloaliphatic or (cyclo)aliphatic diisocyanates as claimed in claim 1,
wherein
the diisocyanates are selected from those having from 3 to 18 carbon atoms.

8. The storage-stable aliphatic, cycloaliphatic or (cyclo)aliphatic diisocyanates as claimed in claim 1,
wherein
the diisocyanates are selected from the group consisting of 1,4-diisocyanotobutane, 2-ethyl-1,4-diisocyanatobutane, 1,5-diisocyanatopentane, 2,2-dimethyl-1,5-diisocyanatopentane, 2-methyl-1,5-diisocyanatopentane (MPDI), 2-ethyl-2-propyl-i,5-diisocyanatopentane, 2-ethyl-2-butyl-1,5-diisocyanatopentane, 2-alkoxy-1,5-diisocyanatopentane, hexamethylene 1,6-diisocyanate (HDI), 2,4,4- or 2,2,4-trimethylhexamethylene 1,6-diisocyanate (TMDI), 1,7-diisocyanatoheptane, 1,8-diisocyanatooctane, 1,10-diisocyanatodecane, 1,12-diisocyanatododecane, 4,4′-diisocyanatodicyclohexylmethane, 2,4′-diisocyanatodicyclohexylmethane and mixtures of the isomeric diisocyanatodicyelohexylmethanes (H12MDI), 1,3-diisocyanatocyclohexane, and isomer mixtures of diisocyanatocyclohexanes and 1-isocyanato-3-isocyanatomethyl-3,5,5-trimethylcyclohexane (IPDI).

9. The storage-stable aliphatic, cycloaliphatic or (cyclo)aliphatic diisocyanates as claimed in claim 8,
wherein
the diisocyanates comprise IPDI, H₁₂MDI, TMDI, HDI and/or MPDI.

10. The storage-stable aliphatic,
cycloaliphatic or (cyclo)aliphatic diisocyanates as claimed in claim 1,
which also contain
stabilizers selected from the group of
a) primary stabilizers and
b) secondary stabilizers.

11. The storage-stable aliphatic, cycloaliphatic or (cyclo)aliphatic diisocyanates as claimed in claim 10,
which contain
a) from 1 to 300 ppm (from 0.0002 to 0.03 mol %) of primary stabilizers or
b) from 1 to 300 ppm (from 0.0002 to 0.03 mol %) of secondary stabilizers.

12. The stable aliphatic, cycloaliphatic or (cyclo)aliphatic diisocyanates as claimed in claim 10,
wherein the primary stabilizers are selected from 2,6-di-tert-butyl-4-methylphenol, 2,4,6-tri-tert-butylphenol, 2,2′-methylenebis(4-methyl-6-tert-butylphenol), 2,2′-thiobis(4-methyl-6-t-butylphenol), 4,4′-thiobis(3-methyl-6-t-butylphenol), 4,4′-butylidenebis(3-methyl-6-tert-butylphenol), 4,4′-methylidenebis(2,6-di-tert-butylphenol), 2,2′-methylidenebis[4-methyl-6-(1-methylcyclohexyl)phenol], tetrakis[methylene 3-(3,5-di-tert-butyl-4-hydroxyphenyl)propionate]methane, 1,3,5-trimethyl-2,4,6-tris(3,5-di-tert-butyl-4-hydroxybenzyl)benzene, N,N′-hexamethylenebis(3,5-di-tert-butyl-4-hydroxyhydrocinnamide), octadecyl 3-(3,5-di-tert-butyl-4-hydroxyphenyl)propionate, 1,3,5-tris(3,5-di-tert-butyl-4-hydroxybenzyl)isocyanurate, 1,1,3-tris(5-tert-butyl-4-hydroxy-2-methylphenyl)-butane, 1,3,5-tris-(3,5-di-tert-butyl-4-hydroxybenzyl)mesitylene; ethylene glycol bis[3,3-bis(3′-tert-butyl-4′-hydroxyphenyl)butyrate], 2,2′-thiodiethyl bis-3-(3,5-di-tert-butyl-4-hydroxyphenyl)propionate, 2,2′-methylenebis(4-methyl-6-cyclohexylphenol), 1,6-hexanediol bis(3,5-di-tert-butyl-4-hydroxyphenyl)propionate, 2,4-bis(n-octylthio)-6-(4-hydroxy-3,5-di-tert-butylanilino)-1,3,5-triazine, diethyl 3,5-di-tert-butyl-4-hydroxybenzylphosphonate and triethylene glycol bis-3-(tert-butyl-4-hydroxy-5-methylphenyl)propionate.

13. The storage-stable aliphatic, cycloaliphatic or (cyclo)aliphatic diisocyanates as claimed in claim 10,
wherein
the secondary stabilizers are selected from esters of phosphorus acid and/or thioethers.

14. The storage-stable aliphatic, cycloaliphatic or (cyclo)aliphatic diisocyanates as claimed in claim 1,
which are blanketed in a storage vessel by an oxygenous atmosphere.

15. The storage-stable aliphatic cycloaliphatic or (cyclo)aliphatic diisocyanates as claimed in claim 10,
which contain
from 1 to 200 ppm (from 0.0007 to 0.14 mol %) of oxygen and from 1 to 200 ppm (from 0.0002 to 0.02 mol %) of a primary stabilizer a) for color reduction under standard conditions.

16. The storage-stable aliphatic, cycloaliphatic or (cyclo)aliphatic diisocyanates as claimed in claim 15,
which contain
from 5 to 100 ppm (from 0.0007 to 0.07 mol %) of oxygen and from 1 to 150 ppm (from 0,0002 to 0.015 mol %) of 2,6-di-tert-butyl-4-methylphenol are present under standard conditions.

17. A process for preparing storage-stable aliphatic, cycloaliphatic or (cyclo)aliphatic diisocyanates, prepared by a phosgene-free process,
which comprises stabilization with oxygen, the oxygen being introduced by means of passing dry air and/or dry oxygen into said diisocyanate.

18. The process as claimed in claim 17,
wherein
the dry air and/or the dry oxygen are introduced into the diisocyanate with a nozzle, a frit or by transposing a turbulent flow.

19. The process as claimed in claim 17,
wherein
the air and/or the oxygen are introduced after the purifying distillation step of the diisocyanate synthesis.

20. The process as claimed in claim 17,
wherein
the air and/or the oxygen is are introduced at temperatures of −20° C. to 200° C., at a pressure of from 5 mbar to 15 bar, with a volume flow rate of from 0.001 to 100 000 liters per hour.

21. The process as claimed in claim 17,
wherein
inert nitrogen or noble gases are used in addition to the air and/or the oxygen.

22. A coating or adhesive raw material comprising storage-stable aliphatic, cycloaliphatic or (cyclo)aliphatic diisocyanates prepared by a phosgene-free process, which also comprises oxygen for stabilization.

23. An aqueous or solvent-containing, liquid or powder coating composition comprising storage-stable aliphatic, cycloaliphatic or (cyclo)aliphatic di- and/or polyisocyanates prepared by a phosgene-free process, which also comprises oxygen for stabilization.

24. (canceled)

25. The storage-stable diisocyanates as claimed in claim 1,
which consist of TMDI stabilized with oxygen and/or air.

26. The storage-stable diisocyanates as claimed in claim 1,
which consist of TMDI and a trialkyl phosphite, stabilized with oxygen and/or air.

27. The storage-stable diisocyanates as claimed in claim 1,
which consist of H₁₂MDI stabilized with oxygen and/or air.

28. The storage-stable diisocyanates as claimed in claim 1,
which consist of H₁₂MDI and 2,6-di-tert-butyl-4-methylphenol stabilized with oxygen and/or air.

29. The storage-stable diisocyanates as claimed in claim 1,
which consist of IPDI stabilized with oxygen and/or air.

30. The storage-stable diisocyanates as claimed in claim 1,
which consist of IPDI and 2,6-di-tert-butyl-4-methylphenol stabilized with oxygen and/or air.
